(19) United States
(12) Patent Application Publication
(10) Pub. No.: US 2007/0286817 A1
(43) Pub. Date: Dec. 13, 2007

(54) Treating cystic fibrosis with antibiotics via a swirler delivery

(75) Inventors: Hanumantharao Tatapudy, Suffern, NY (US); Syed M. Shah, East Hanover, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/810,182

(22) Filed: Jun. 5, 2007

Related U.S. Application Data
(60) Provisional application No. 60/811,671, filed on Jun. 7, 2006.

(51) Int. Cl. A61K 9/12; A61K 31/43; A61K 31/545

(52) U.S. Cl. 424/45; 514/192; 514/200

(57) ABSTRACT

The present invention is directed to a method of treating respiratory disorders by delivering an aerosol composition of an antibiotic drug to the lung alveoli.

This application claims priority from co-pending U.S. provisional application No. 60/811,671 filed on Jun. 7, 2006.

### BACKGROUND OF THE INVENTION

The present invention is directed to a method of treating respiratory disorders by delivering an antibiotic aerosol formulation to the lung alveoli.

Nebulizers and other aerosol devices have been used to deliver drugs for asthma patients. However, not all these devices deliver droplets that are small enough to provide deep lung penetration, which may be beneficial in delivering certain drugs, such as antibiotics. Some aerosol devices are capable of delivering the smaller droplet sizes needed for deep lung penetration. One commercially available example is the SWIRLER® aerosol drug delivery system which is described at amici-inc.com, and in U.S. Pat. Nos. 5,603,314, 5,630,409, 5,611,332 and 6,230,703, which patents are incorporated by reference herein.

As described in greater detail in the aforementioned patents, the SWIRLER® aerosol drug delivery system is an aerosol inhalation device that provides an aerosol mist to a patient. This device includes a nebulizer having a liquid reservoir containing the liquid to be inhaled, a gas inlet for receiving pressurized gas, and an aerosol outlet. An important feature of the device is a gas swirling or flow control means which creates a swirling action to the gas forming the aerosol; this produces a greater shear force and smaller particle sizes. The swirling gas creates a vacuum as it exits the outlet and this vacuum draws liquid form the reservoir, producing an aerosol. The device is capable of producing aerosol particles less than one micrometer in size.

ZOSYN® is an injectable antibacterial combination product consisting of the semi synthetic antibiotic piperacillin sodium and the (beta)-lactamase inhibitor tazobactam sodium for intravenous administration. The product is disclosed in U.S. Pat. Nos. 4,562,073, 4,477,452, 4,534,977, and 6,207,661.

Piperacillin sodium is derived from D(−)-(alpha)-aminobenzyl-penicillin. The chemical name of piperacillin sodium is sodium (2S,5R,6 R)-6-[(R)-2-(4-ethyl-2,3-dioxo-1-piperazine-carboxamido)-2-phenylacetamido]-3,3-dimethyl-7-oxo-4-thia-1-azabicyclo[3.2.0]heptane-2-carboxylate. The chemical formula is C₂₃H₂₆N₅NaO₇S and the molecular weight is 539.5. The product is disclosed in U.S. Pat. No. 4,562,073.

The chemical structure of piperacillin sodium is:

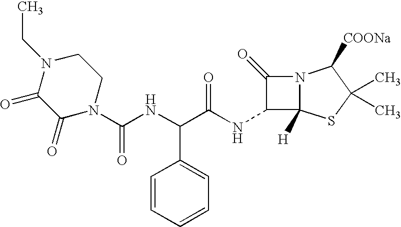

Tazobactam sodium, a derivative of the penicillin nucleus, is a penicillanic acid sulfone. Its chemical name is sodium (2S,3 S,5 R)-3-methyl-7-oxo-3-(1H-1,2,3-triazol-1-ylmethyl)-4-thia-1-azabicyclo[3.2.0]heptane-2-carboxylate-4,4-dioxide. The chemical formula is C₁₀H₁₁N₄NaO₅ S and the molecular weight is 322.3. The product is disclosed in U.S. Pat. No. 4,958,020.

The chemical structure of tazobactam sodium is:

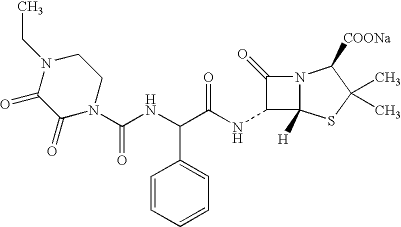

TYGACIL® (tigecycline) is a first in class glycylcycline antibacterial disclosed in U.S. Pat. No. 5,494,903. The chemical name of tigecycline is (4S,4aS,5aR,12aS)-9-[2-(tert-butylamino)acetamido]-4,7-bis(dimethylamino)-1,4,4a,5,5a,6,11,12a-octahydro-3,10,12,12a-tetrahydroxy-1,1′-dioxo-2-naphthacenecarboxamide. The empirical formula is C₂₉H₃₉N₅O₈ and the molecular weight is 585.65. It is a 9-tert-butyl-glycylamido derivative of monocycline which exhibits antibiotic activity typical of tetracyclines, but has more potent activity against tetracycline-resistant organisms having efflux and ribosomal protection mechanisms of resistance. Tigecycline has an expanded spectrum of activity against gram positives, gram negatives, anaerobes, and atypicals including resistant pathogens, and allows for flat dosing. The product is disclosed in U.S. Pat. Nos. 5,494,903, 5,299,900, and 5,284,963.

The following represents the chemical structure of tigecycline:

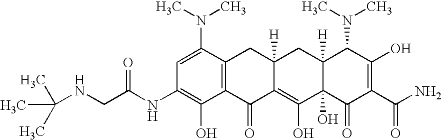

There exists a need to deliver these and other antibiotics to patients via aerosol directly to the lungs to provide another option in the treatment of lung disorders, for example, cystic fibrosis.

### SUMMARY OF THE INVENTION

These and other embodiments are provided for by the invention disclosed and claimed herein.

The present invention comprises a method of treating a respiratory disorder comprising administering an antibiotic drug suitable for treating said disorder to a mammal in need thereof via a drug delivery system such as the SWIRLER aerosol drug delivery system, which produces an aerosol composition of said antibiotic drug in which the particle size of the antibiotic composition droplets is small enough to provide deep lung penetration. Preferably, at least about 90%, such as 95% or more, of the antibiotic droplets in the aerosol are about 1-3 microns or less, more preferably 1.1 microns or less.

For delivery, the antibiotic aerosol typically will comprise an antibiotic and a diluent. The diluent can be, for example, sterile water for Injection, 0.9% sodium chloride for injection, 5% dextrose for injection, 5% dextrose and 0.9% sodium chloride for injection, 5% dextrose in lactated Ringers for injection, 5% dextrose-0.45% sodium chloride-0.15% potassium chloride for injection or lactated Ringers for injection.

In the practice of this invention, a liquid composition comprising the drug and the diluent is placed in the reservoir of the SWIRLER® aerosol drug delivery system, which is connected to a source of pressurized gas, which gas is not reactive with the liquid composition. The device is designed to impart a swirling action to the gas and to create a fine mist of aerosol droplets which can be smaller than one micrometer.

Antibiotics of the present invention include anti-infective agents known in the art, such as those found in the current Physician's Desk Reference published by Medical Economics Company (www.pdr.net) and hereby incorporated by reference, including but are not limited to ZOSYN®, Piperacillin, Tazobactam, and TYGACIL®. The antibiotic can be administered alone or in combination with other antibiotics. In accordance with the invention, at least one of the antibiotics is administered in an aerosol medium composition. Additional antibiotics may be administered orally, or by intralesional, intraperitoneal, intramuscular or intravenous injection; infusion; liposome-mediated delivery; topical, nasal, anal, vaginal, sublingual, uretheral, transdermal, intrathecal, ocular or optic delivery. In order to obtain consistency in providing the compound of this invention it is preferred that a compound of the invention is in the form of a unit dose. Suitable unit dose forms include tablets, capsules and powders in sachets or vials. Such unit dose forms may contain from 0.1 to 300 mg of a compound of the invention and preferably from 2 to 100 mg. Still further preferred unit dosage forms contain 5 to 50 mg of a compound of the present invention. The effective amount will be known to one of skill in the art; it will also be dependent upon the form of the compound. One of skill in the art could routinely perform empirical activity tests to determine the bioactivity of the compound in bioassays and thus determine what dosage to administer.

### DETAILED DESCRIPTION OF THE INVENTION

In one embodiment of the present invention, there is provided a method of delivering an aerosol medium composition containing an antibiotic via the SWIRLER drug delivery system. The aerosol medium composition may comprise diluents such as sterile water for injection, 0.9% sodium chloride for injection, 5% dextrose for injection, 5% dextrose and 0.9% sodium chloride for injection, 5% dextrose in lactated Ringers for injection, 5% dextrose-0.45% sodium chloride-0.15% potassium chloride for injection or lactated Ringers injection.

In one embodiment, the antibiotic is suspended in the aerosol medium at a particle size range that will meet the subvisible particulate testing acceptance criteria as per USP 788 viz. not more than 600 particles ≧25 microns and not more than 6000 particles ≧10 microns.

In one embodiment, the invention comprises a method of treating respiratory disorders using the SWIRLER drug delivery system to deliver an aerosol containing an antibiotic compound and to reduce the particle size of at least about 95% of the antibiotic droplets in the aerosol to about 1-3 microns or less, thereby allowing the antibiotic aerosol to reach the alveoli of the lung. In another embodiment, at least about 95% of the antibiotic particles is delivered at a particle size of less than 1.1 microns.

This method is useful for treating a respiratory disorder such as, but not limited to, cystic fibrosis.

When used herein, the term “about” shall generally mean within 20 percent.

It will be understood by those with skill in the art that the invention may be performed within a wide and equivalent range of conditions, parameters and the like, without affecting the spirit or scope of the invention or any embodiment thereof.

A drug such as Piperacillin (2 g-4 g lyophilized powder per vial), Tazobactam (0.25 g-0.50 g lyophilized powder per vial), Tygacil (50 mg lyophilized powder per 5 mL vial), or ZOSYN® (2-4 g piperacillin plus 250-500 mg tazobactam), and at least one intravenous diluent, for example but not limited to sterile water for injection, 0.9% sodium chloride injection, 5% dextrose injection, 5% dextrose and 0.9% sodium chloride injection, 5% dextrose in Lactated Ringers injection, 5% dextrose-0.45% sodium chloride-0.15% potassium chloride injection or lactated Ringers injection may be administered using a SWIRLER drug delivery system, or an equivalent aerosol delivery system, at a particle size of 95% particles ranging from about 1 to about 3 microns and preferably less than about 1.1 microns to ensure deep lung delivery to the alveolar region of the lungs. Those skilled in the art will readily be capable of determining whether a delivery system is able to provide the aerosol particle sizes of the present invention.

The following examples are presented to illustrate certain embodiments of the present invention, but should not be construed as limiting the scope of this invention.

### EXAMPLE

### Tygacil® (Tigecycline) for Deep Lung Delivery

The commercial Tygacil® 2^{nd} Generation product was used to conduct the study. Sterile Water for injection and 0.9% Normal Saline were used at diluents. Tygacil® is a sterile, lyophilized powder for intravenous infusion, containing 53 mg of the Tigecycline active ingredient. Tygacil® additionally contains lactose monohydrate as a diluent/stabilizer and hydrochloric acid and/or sodium hydroxide (as needed) for pH adjustment. The product is supplied in a single dose; Type I, clear, glass vial, sealed under a blanket of nitrogen with a gray butyl rubber stopper and a snap-off aluminum crimp seal.

### The Quantitative Composition of Tygacil® is Depicted in Table 1 Below

TABLE 1
Quantitative Composition for Tygacil ®

| | Reference to | | |
|---|---|---|---|
| Ingredient | Standards | Function | Quantity per Vial |
| Tigecycline^{a} | In-House | Active | 53 mg |
| | Monograph | | |
| Lactose | NF/Ph. Eur.^{b} | Diluent/ | 106 mg |
| Monohydrate | | Stabilizer | |
| Hydrochloric Acid | NF/Ph. Eur. | pH Adjustment | Q.S. to adjust pH |
| Sodium Hydroxide | NF/Ph. Eur. | pH Adjustment | Q.S. to adjust pH |
| Water for Injection^{c} | USP/Ph. Eur. | Vehicle | —^{c} |
| Nitrogen^{d} | NF/Ph. Eur. | Blanket | Q.S. to |
| | | | Headspace |

^{a}A 6% overage is included to compensate for the non-withdrawable amount of solution after constitution, i.e. solution adhering to the inside wall of the vial.
^{b}An in-house specification for bacterial endotoxins test is also applied.
^{c}Removed during lyophilization.
^{d}Used for sparging and blanketing the bulk solution and as inert cover in the filled vials.

Prior to reconstitution, Tigecycline for Injection is an orange powder or cake. One (1) vial of Tygacil® was reconstituted using 100 ml of 0.9% Sodium Chloride (Normal Saline) or Sterile Water for Injection USP. The Tigecycline powder was allowed to dissolve in the diluents. A clear yellow to orange solution was obtained. The solution was then transferred into the SWIRLER® device. Oxygen was supplied to the SWIRLER® via an NG tube to aerosolize the solution. An oxygen air pressure regulator was used to set the air pressure to 15 CFM. A Malvern MXS, S/N 6196 was used to measure the particle size of the droplets. Results show a high percentage (90%) of the particles are less than 1.1 micron which is the desired size for deep lung delivery. Data are reported in Table 2 below:

TABLE 2
PARTICLE SIZE DISTRIBUTION OF TYGACIL ®
RECONSTITUTED WITH NORMAL SALINE OR STERILE
WATER FOR INJECTION AND ADMINISTERED
THROUGH A SWIRLER ® FOR DEEP LUNG DELIVERY

| | Obscuration | D 10 | D 50 | D 90 |
|---|---|---|---|---|
| | (%) | (um) | (um) | (um) |
| Water | | | | |
| Trial 1 | 32.7 | 0.38 | 0.58 | 0.93 |
| Trial 2 | 32.7 | 0.41 | 0.61 | 0.99 |
| Trial 3 | 36.7 | 0.37 | 0.56 | 0.88 |
| 0.9% NaCl | | | | |
| Trial 1 | 28.1 | 0.42 | 0.62 | 1.02 |
| Trial 2 | 31 | 0.4 | 0.6 | 0.98 |
| Trial 3 | 33.9 | 0.4 | 0.6 | 0.98 |
| 0.9% NaCl + TYG* | | | | |
| Trial 1 | 27.8 | 0.41 | 0.61 | 1 |
| Trial 2 | 16.6 | 0.39 | 0.59 | 0.96 |
| Trial 3 | 14.9 | 0.38 | 0.58 | 0.94 |
| 0.9% NaCl + | | | | |
| TYG** | | | | |
| Trial 1 | 28 | 0.45 | 0.65 | 1.11 |
| Trial 2 | 25.2 | 0.44 | 0.64 | 1.1 |
| Trial 3 | 30.4 | 0.44 | 0.65 | 1.08 |
| Water + TYG*** | | | | |
| Trial 1 | 32.9 | 0.42 | 0.62 | 1.01 |
| Trial 2 | 30.6 | 0.41 | 0.61 | 0.99 |
| Trial 3 | 33.5 | 0.41 | 0.61 | 1 |

*100 ml of 0.9% Sodium Chloride (Normal Saline) was used to dissolve 50 mg (1 vial) of Tygacil ®
**100 ml of 0.9% Sodium Chloride (Normal Saline) was used to dissolve 100 mg (2 vials) of Tygacil ®
***100 ml of Sterile Water for Injection (USP) was used to dissolve 50 mg (1 vial) of Tygacil ®

Many variations of the present invention not illustrated herein will occur to those skilled in the art. The present invention is not limited to the embodiments illustrated and described herein, but encompasses all the subject matter within the scope of the appended claims.

What is claimed is:

1. A method of treating a respiratory disorder comprising administering an antibiotic drug suitable for treating said disorder to a mammal in need thereof via a SWIRLER drug delivery system which produces a liquid aerosol composition of said antibiotic drug in which the particle size of at least about 90% of the antibiotic droplets in the aerosol is about 1-3 microns or less, wherein said antibiotic drug comprises ZOSYN, Piperacillin, Tazobactam, or TYGACIL.

2. The method of claim 1, wherein the particle size of at least about 95% of the aerosol composition is 1-3 microns or less.

3. The method of claim 1, wherein the particle size of at least about 90% of the antibiotic droplets in the aerosol is about 1.1 microns or less.

4. The method of claim 1, wherein the particle size of at least about 95% of the antibiotic droplets in the aerosol is about 1.1 microns or less.

5. The method of claim 1, wherein the aerosol composition comprises an antibiotic and a diluent.

6. The method of claim 5, wherein the diluent comprises sterile water for Injection, 0.9% sodium chloride for injection, 5% dextrose for injection, 5% dextrose and 0.9% sodium chloride for injection, 5% dextrose in lactated Ringers for injection, 5% dextrose-0.45% sodium chloride-0.15% potassium chloride for injection, or lactated Ringers for injection.

7. The method of claim 1, wherein the respiratory disorder is cystic fibrosis.

8. The method of any one of claims 1, wherein the antibiotic can be administered alone or in combination with other antibiotics.

9. The method of claim 1, wherein the particle size of at least about 90% of the antibiotic droplets in the aerosol is about 1.1 microns or less and the aerosol composition comprises an antibiotic and a diluent.

10. The method of claim 9, wherein the respiratory disorder is cystic fibrosis.
